(19) United States
(12) Patent Application Publication
(10) Pub. No.: US 2025/0012768 A1
(43) Pub. Date: Jan. 9, 2025

(54) OZONE SENSOR WITH OFF-STOCHEOMETRIC DELAFOSSITE-TYPE COPPER OXIDE

(71) Applicant: LUXEMBOURG INSTITUTE OF SCIENCE AND TECHNOLOGY (LIST), ESCH-SUR-ALZETTE (LU)

(72) Inventors: Joao Ricardo ANTUNES AFONSO, ESCH-SUR-ALZETTE (LU); Damien LENOBLE, ESCH-SUR-ALZETTE (LU)

(21) Appl. No.: 18/562,578

(22) PCT Filed: May 4, 2022
(86) PCT No.: PCT/EP2022/061921

§ 371 Date: Nov. 20, 2023

(30) Foreign Application Priority Data
May 19, 2021 (LU) LU500174

(51) Int. Cl. G01N 33/00; G01N 27/12; G01N 27/18

(52) U.S. Cl. CPC G01N 33/0039; G01N 27/125; G01N 27/18

(57) ABSTRACT

A gas sensor comprising a substrate; a layer of delafossite-type copper oxide on the substrate; a first electrode and a second electrode, both contacting the delafossite-type copper oxide at distant locations so as to permit an electric current through said delafossite-type copper oxide between said locations when applying a voltage at said first and second electrodes; wherein the delafossite-type copper oxide is Cu_{0.66}Cr_{1.33}O₂.

### CROSS-REFERENCE TO RELATED APPLICATIONS

The present invention is the US national stage under 35 U.S.C. § 371 of International Application No. PCT/EP2022/061921 which was filed on May 4, 2022, and which claims the priority of application LU500174 filed on May 19, 2021 the contents of which (text, drawings and claims) are incorporated here by reference in its entirety.

### FIELD

The invention is directed to the field of gas sensing, more particularly gas sensing with a p-type semiconductor, even more particularly ozone sensing with a delafossite-type copper oxide.

### BACKGROUND

Shu Zhou, Xiaodong Fang, Zanhong Deng, Da Li, Weiwei Dong, Ruhua Tao, Gang Meng, Tao Wang, Room temperature ozone sensing properties of p-type CuCrO2 nanocrystals, Sensors and Actuators B: Chemical, Volume 143, Issue 1, 2009, Pages 119-123, ISSN 0925-4005, https://doi.org/10.1016/snb.2009.09.026, discloses ozone sensors comprising a layer of delafossite-type copper oxide CuCrO₂ synthetized by hydrothermal and sol-gel methods. That ozone sensor is operated at room temperature contrary to n-type semiconductor gas sensors which require operation at temperatures of more than 200° C. However, that ozone sensor is able to detect ozone only in the ppm range, i.e., from 50 ppm. It is however in practise often desirable to detect ozone in lower concentrations, i.e., in the ppb (parts-per billion) range.

Shamatuofu Bai, Sheng-Chi Chen, Song-Sheng Lin, Qian Shi, Ying-Bo Lu, Shu-Mei Song, Hui Sun, Review in optoelectronic properties of p-type CuCrO2 transparent conductive films, Surfaces and Interfaces, Volume 22, 2021, 100824, ISSN 2468-0230, https://doi.org/10.1016/j.surfin.2020.100824, is a general review of the optoelectronic properties of p-type CuCrO₂ transparent conducive films. Among others, the gas sensitivity is briefly discussed. Under ozone ambient, the resistance of the ozone sensor containing CuCrO2 nanocrystals/microcrystals decreases, while the resistance almost returns to its original value as the ozone gas is removed. The ozone sensitivity of CuCrO2 originates from the extra hole concentration in the enriched area of the surface with the presence of ozone gas. With reference to the above citation, CuCrO₂ can be prepared through hydrothermal and sol-gel methods. The films express good reversible response to the ozone gas at room temperature.

### SUMMARY

The invention has for technical problem to overcome at least one of the drawbacks of the above cited prior art. More specifically, the invention has for technical problem to provide a gas sensor, for instance an ozone sensor, showing a higher sensitivity, in particular in the ppb range, a good selectivity, notably with regard to oxygen (in molecular form O₂), and/or operating at room or limited temperatures.

The invention is directed to a gas sensor comprising a substrate; a layer of delafossite-type copper oxide on the substrate; a first electrode and a second electrode, both contacting the delafossite-type copper oxide at distant locations so as to permit an electric current through said delafossite-type copper oxide between said locations when applying a voltage at said first and second electrodes; wherein the delafossite-type copper oxide is Cu_{0.66}Cr_{1.33}O₂.

According to an exemplary embodiment, the gas sensor is an ozone sensor.

According to an exemplary embodiment, the delafossite-type copper oxide shows a conductivity that increases when in contact with ozone.

According to an exemplary embodiment, the gas sensor is configured for operating at a constant temperature comprised between 25° and 150° C.

According to an exemplary embodiment, the delafossite-type copper oxide shows an outer surface average roughness of at least 1 nm, in various instances at least 2 nm, for example at least 5 nm.

According to an exemplary embodiment, the delafossite-type copper oxide shows an outer surface average roughness of not more than the layer thickness.

Advantageously, the delafossite-type copper oxide shows an outer surface average roughness of not more than 10 nm.

According to an exemplary embodiment, the layer of delafossite-type copper oxide shows an average thickness of at least 20 nm, various instances 30 nm.

According to an exemplary embodiment, the layer of delafossite-type copper oxide shows an average thickness of not more than 200 nm, various instances 250 nm.

According to an exemplary embodiment, the delafossite-type copper oxide is annealed, various instances at a temperature comprised between 800° and 1200° C.

According to an exemplary embodiment, the gas sensor further comprises a heater provided on the substrate opposite to the layer of delafossite-type copper oxide.

According to an exemplary embodiment, the substrate is a dielectric layer of a microheater system.

According to an exemplary embodiment, the gas sensor is configured for detecting ozone in a concentration range from 10 to 100 000 ppb

The invention is also directed to a method of measuring the presence and/or concentration of ozone, comprising using a gas sensor with a substrate, a layer of delafossite-type copper oxide on the substrate, and electrodes on the delafossite-type copper oxide at distant locations, contacting the delafossite-type copper oxide with the ozone, and electrically measuring a variation of resistance of the delafossite-type copper oxide; wherein the gas sensor is according to the invention.

According to an exemplary embodiment, during contacting and measuring, the layer of delafossite-type copper oxide is maintained at a constant temperature comprised between 25° and 150° C.

According to an exemplary embodiment, the method further comprises: determining a slope of the variation of resistance of the delafossite-type copper oxide and deducting from said slope the concentration of ozone.

According to an exemplary embodiment, the slope of the variation of resistance of the delafossite-type copper oxide is determined over a period of time comprised between 20 and 200 seconds from contacting the delafossite-type copper oxide with the ozone.

The invention is also directed to a method for manufacturing a gas sensor, comprising the following steps: providing a substrate; depositing a layer of delafossite-type copper oxide on the substrate; depositing electrodes on the delafossite-type copper oxide; wherein the gas sensor is according to the invention.

According to an exemplary embodiment, the method comprises the further step of, after depositing the layer of delafossite-type copper oxide on the substrate: annealing the layer of delafossite-type copper oxide.

According to an exemplary embodiment, the annealing step is carried out at a temperature comprised between 800° and 1200° C.

According to an exemplary embodiment, the annealing step is carried out by laser scanning.

The invention is particularly interesting in that it provides an ozone gas sensor with a good sensitivity and selectivity at low concentrations of ozone and operating at temperatures of less than 150° C., including room temperature of 25° C. This substantially reduces the power consumption of the gas sensor in that no heating unit is necessary, or if one is needed, it shows a reduced nominal power, size, cost and power consumption, compared with gas sensors operating at temperatures substantially above 150° C., as in the prior art.

### DRAWINGS

FIG. 1 is a schematic section view of a gas sensor according to various embodiments of the invention.

FIG. 2 illustrates graphically the operational temperature ranges of various solid-state oxides used for gas detection, as well as the operational temperature range of the solid-state oxide according to various embodiments of the invention.

FIG. 3 illustrates graphically the current passing through the gas sensor of the invention over time in the presence of a varying ozone concentration according to various embodiments of the invention.

FIG. 4 illustrates graphically the current passing through the gas sensor of the invention over time in the presence of different constant ozone concentrations according to various embodiments of the invention.

FIG. 5 is based on FIG. 4 and illustrates graphically the current passing through the gas sensor of the invention over a shorter time in the presence of the different constant ozone concentrations according to various embodiments of the invention.

FIG. 6 illustrates graphically the current passing through the gas sensor of the invention in the presence, successively, of different gases, showing the selectivity of the gas sensor, in particular against molecular oxygen present in air according to various embodiments of the invention.

FIG. 7 shows Scanning Electron Microscope top views of layers of delafossite-type copper oxide of the gas sensor of the invention, with a thickness of 140 nm and 32 nm and as deposited, annealed at 900° C. and annealed at 1050° C. according to various embodiments of the invention.

### DETAILED DESCRIPTION

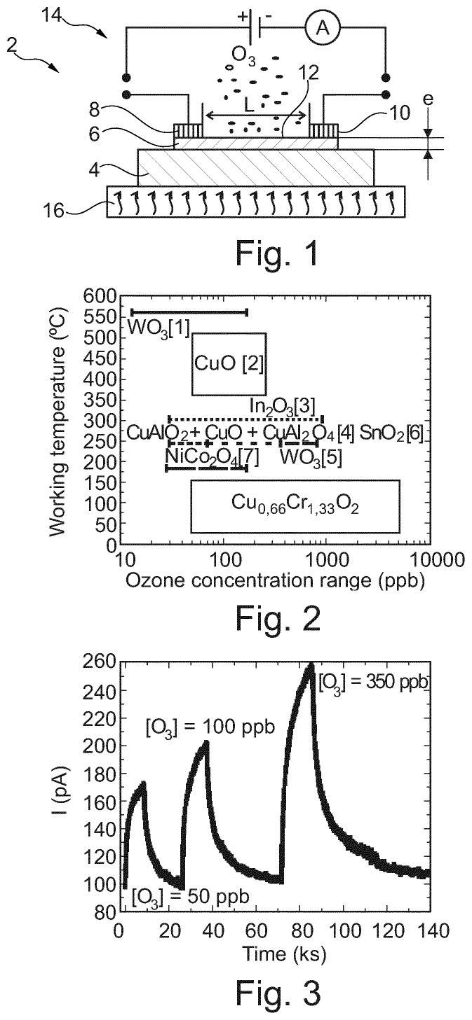

FIG. 1 is a schematic sectional view of a gas sensor according to various embodiments the invention.

The gas sensor 2 comprises essentially a substrate 4, a layer of delafossite-type copper oxide 6 deposited on the substate 4, and at least two electrodes 8 and 10 provided on the layer of delafossite-type copper oxide 6, at distant locations so as to form a sensing area 12 between the electrodes 8 and 10.

The gas sensor 2 can be electrically connected to a measuring circuit 14 that is also schematically represented in FIG. 1, comprising essentially a voltage power supply and an amperemeter. The layer of delafossite-type copper oxide 6 between the electrodes 8 and 10 has an electric resistance R_{L} that varies in contact with ozone O₃, as this will be explained here after. Each electrode 8 and 10 can also feature an electric resistance R_{E}, essentially resulting of the contact with the layer of delafossite-type copper oxide 6. By applying a voltage, e.g., a direct voltage, to the layer of delafossite-type copper oxide 6 between the electrodes 8 and 10, the current I=V/(R_{L}+2R_{E}), measured with the amperemeter, will vary as the electric resistance R_{E} varies. In other words, the measuring circuit 14 described here above in a simplistic manner allows to detect and/or measure the presence of ozone or a variation of concentration thereof in contact with the sensing area 12 of the layer of delafossite-type copper oxide 6.

The delafossite-type copper oxide 6 is specifically an off-stoichiometric Cu—Cᵣ−O delafossite, i.e., Cu₁₋ᵤCr₁₊ᵤO₂ where 0<u<1, advantageously u=0.33. Among delafossite materials, it is focused on CᵤCᵣO₂ due to its high density of 3d cations near the maximum of valence band and the covalent mixing between chromium and oxygen ions. These two properties promote larger holes mobility and therefore a greater conductivity. In practice however, CᵤCᵣO₂ shows a low conductivity (10⁻⁴ S cm⁻¹) so that doping is useful for increasing the conductivity to more than at least 1 S cm⁻¹. Off-stoichiometry as mentioned above further increases the conductivity. For instance, Cu_{0.66}Cr_{1.33}O₂ shows a conductivity of about 10² S cm⁻¹.

The electrical properties of off-stoichiometric Cu—Cr—O delafossite are largely discussed and analyzed in Lunca-Popa, P., Botsoa, J., Bahri, M. et al. Tuneable interplay between atomistic defects morphology and electrical properties of transparent p-type highly conductive off-stoichiometric Cu—Cr—O delafossite thin films. Sci Rep 10, 1416 (2020). https://doi.org/10.1038/s41598-020-58312-z.

The inventors of the present invention have found that the above off-stoichiometric Cu—Cᵣ—O delafossite shows a strong catalytic activity with ozone decomposition into 3 monoatomic oxygens, as per the following equation.

(O₃)gas+3e⁻→3O⁻+3h⁺

This allows the off-stoichiometric Cu—Cᵣ—O delafossite material to achieve a reversible measurement of ozone, in particular at low concentrations, i.e., in the ppb range, and at low temperature, i.e., less than 150° C.

The gas sensor 2 illustrated in FIG. 1 can comprise a heating plate 16 provided against the substrate 4 on a main face thereof that is opposite to the layer of delafossite-type copper oxide 6. The heating plate 16 can be operated so as to maintain the substrate 4 and more particularly the layer of delafossite-type copper oxide 6 at a constant temperature, i.e., within a determined and limited temperature range.

Still with reference to FIG. 1, the layer of delafossite-type copper oxide 6 shows an average thickness e that is advantageously of at least 30 nm. The sensing area can show a length L between the electrodes 8 and 10 that is at least 250 nm and/or not more than 1000 nm.

FIG. 2 illustrates in graphical and schematic manner the operating ranges in temperature and ozone concentrations of different known materials. For instance, WO₃, as reported in Utembe et al, 2006 Sensors and Actuators B 114, operates at temperatures of more than 550° C. for ozone concentrations ranging from 10 to about 100 ppb. Also, CᵤO₂, as reported in SsBejaoui et al, 2014 Sensors and Actuators B 190, operates at temperatures comprised between 350 and 500° C. for ozone concentrations of about 100 ppb. Further In₂O₃, as reported in Korotcenkov et al, Journal of sensors 2016 operates at temperatures of about 300° C. Still further, CuAlO₂+CuO+CuAl₂O₄, as reported in Baratto et al, 2014 Sensors and Actuators B 20, operates at temperatures of about 250° C. Similarly, WO₃, as reported in Guerin et al, 2008 Sensors and Actuators B 128, operates also at 250° C. The material SnO₂, as reported in Korotcenkov et al, 2007 Sensors and Actuators B 120, operates between 25° and 300° C. for concentrations of about 1000 ppb. Last, NiCo₂O₄, as reported in Joshi et al., 2016 RSC Advances 95, operates at temperatures of about 200° C.

As this is apparent in FIG. 1, the delafossite-type copper oxide Cu_{0.66}Cr_{1.33}O₂ operates at a constant temperature comprised between 25° and 150° C., i.e., substantially lower than the above listed materials and for ozone concentrations comprised between 50 and 5000 ppb (i.e., 5 ppm).

FIGS. 3 to 5 illustrate the electrical behaviour of the gas sensor of the invention. The graphics in these figures result from experimental measurements made at 100° C. with a gas sensor whose layer of delafossite-type copper oxide Cu_{0.66}Cr_{1.33}O₂ shows a thickness e of 32 nm and which has been annealed at 1050° C.

FIG. 3 is a graphic showing the current flowing through the gas sensor of the invention, as illustrated in FIG. 1, when the gas sensor is contacted successively by ozone concentrations of 50 ppb, 100 ppb and 350 ppb. We can observe that the current shows different rising profiles depending on the ozone concentration. With the first ozone concentration of 50 ppb, the current rises from about 100 pA to about 170 pA after about 10 ks, whereas with the second ozone concentration of 100 ppb, the current rises from about 100 pA to about 200 pA after about the same period of 10 ks, and with the third ozone concentration of 350 ppb, the current rises up to 260 pA after about the same period of 10 ks. This behaviour demonstrates the sensitivity of the gas sensor to different ozone concentrations within a lower range for instance of 50 to 350 ppb.

FIG. 3 shows however very long periods of time of 10 ks, i.e., about 2 hours and 47 minutes, which might in certain applications not be practicable.

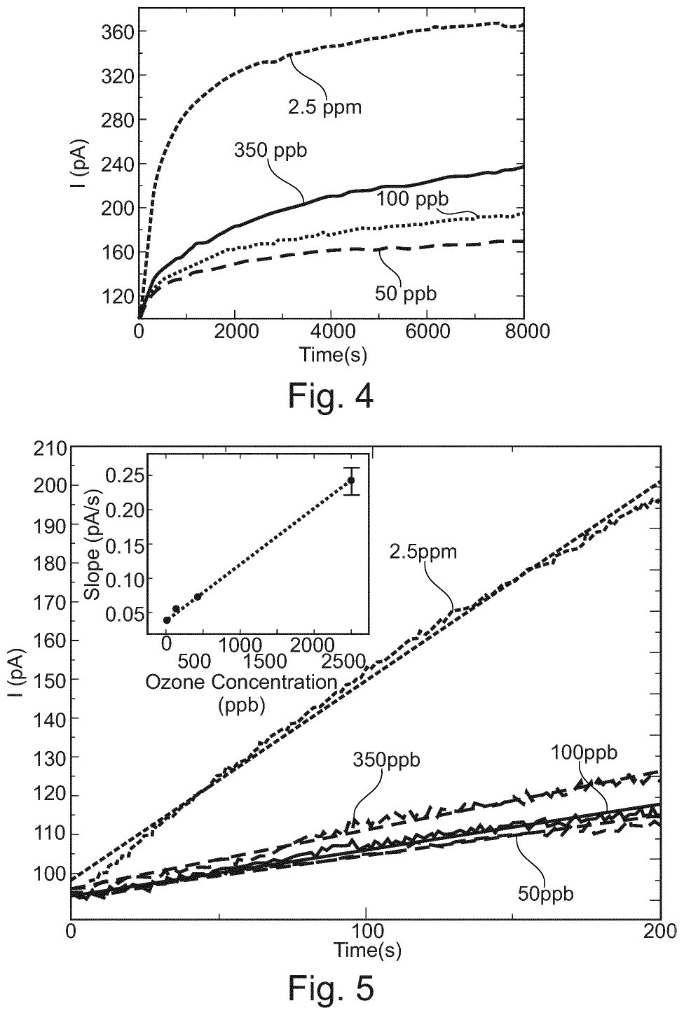

FIG. 4 is a graphic showing the current flowing through the gas sensor of the invention, as illustrated in FIG. 1, when the gas sensor is contacted by ozone at different concentrations. Contrary to the FIG. 3, these different concentrations are not reported in a successive manner as in FIG. 3 but rather over the same time period, for instance of 8000 seconds. In other words, the time scale in FIG. 4 is substantially shorter as in FIG. 3 and also the current behaviours at the different ozone concentrations are reported in parallel.

We can observe that saturation in the current behaviour occurs after 2000 s from the moment when the specific ozone concentration is applied to the gas sensor and also, similarly as in FIG. 3, that the saturated current values are quite different for the different ozone concentrations. We can also observe that the slopes of the current at the very beginning of application of the ozone concentrations to the gas sensor are also quite different.

FIG. 5 is based on FIG. 4, showing the current flowing through the gas sensor of the invention at the different ozone concentrations, however during a reduced period of time of 200 s from the very beginning of application of the different ozone concentrations to the gas sensor. We can observe that the current changes over time are nearly straight and can be approximated to a linear function. We can also observe that these linear functions show different slopes at the different ozone concentrations.

The current slopes (over time, expressed in pA/s) for the four ozone concentrations of 50 ppb, 100 ppb, 350 ppb and 2500 ppb (i.e., 2.5 ppm) are reported in a sub-graphic embedded in the main graphic. We can observe that the current slope over the ozone concentration can be approximated to a linear function.

The above-described FIGS. 3 to 5 show the advantages of the electric behaviour of the gas sensor of the invention, i.e., in terms of ozone concentration sensitivity as well as reactivity by determining the current slope during measurement over a limited period of time, and deducting the ozone concentration from that slope.

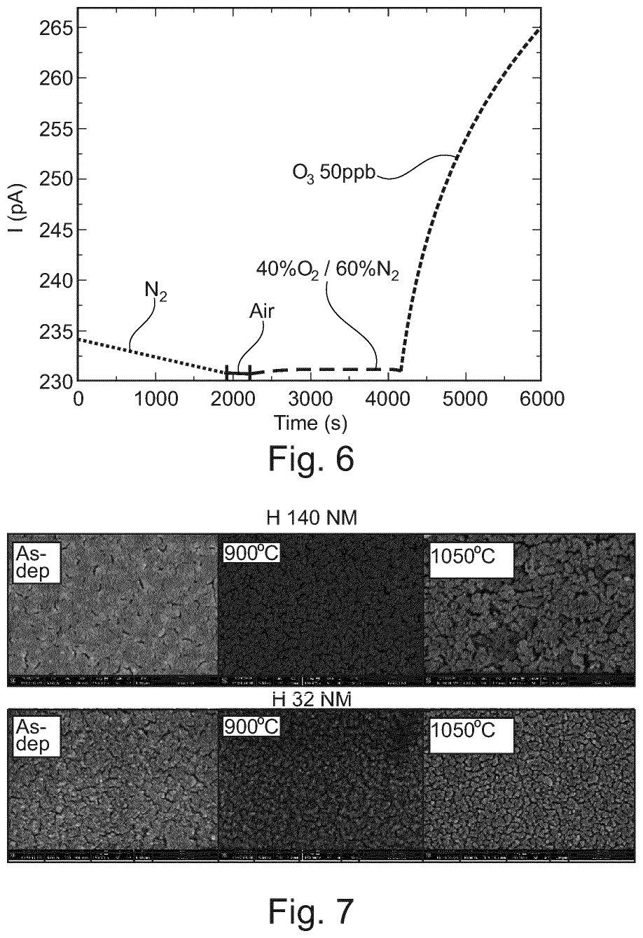

FIG. 6 is a further graphic showing the current flowing through the gas sensor of the invention, over time when contacted successively by different gases, for instance by nitrogen (N₂), air, a mixture of 40% O₂ and 60% N₂, and ozone (O₃). This figure results from experimental measurements made at 100° C. with a gas sensor whose layer of delafossite-type copper oxide Cu_{0.66}Cr_{1.33}O₂ shows a thickness e of 32 nm and which has been annealed at 1050° C.

As this is apparent, during contact of the gas sensor first with gaseous nitrogen, for instance during about 1900 s, the current decreases from about 234 pA to about 231 pA. Thereafter, the gas sensor is contacted with air during about 250 s whereby the current shows no significant change. The gas sensor is then contacted with the mixture of 40% O₂ and 60% N₂ during about 1900 s where the current only very slightly increases of less than 1 pA. The gas sensor is then contacted by ozone at a concentration of 50 ppb, i.e., a low concentration, where the current immediately and drastically increases in comparison with the previous variations when in contact with the other gases. This demonstrates the high selectivity of the gas sensor to ozone even with a lower ozone concentration, e.g., 10 ppb.

The above-described layer of delafossite-type copper oxide can be deposited by Chemical Vapor Deposition (CVD), using copper and chromium precursors and a flow of oxygen. That deposition typically is at temperatures comprised between 30° and 500° C. The deposition can be as detailed in P. Lunca Popa, J. Crêpellière, R. Leturcq, D. Lenoble, Electrical and optical properties of Cu—Cr—O thin films fabricated by chemical vapour deposition, Thin Solid Films, Volume 612, 2016, Pages 194-201, ISSN 0040-6090, https://dol.org/10.1016/j.tsl2016.05.052.

Before deposition of the layer of delafossite-type copper oxide can be deposited, a mask can be formed on the substrate, so as to control the area(s) of the substrate onto which the layer is deposited. After deposition, the mask can be removed. Application and removal of such a mask is as such well known to the skilled person.

The substrate can be a dielectric layer of a microheater system.

The above-described layer of delafossite-type copper oxide, after being deposited on a substrate, is advantageously annealed at temperatures comprised between 800° and 1200° C. The annealing can be carried out in a reactor with the same gaseous conditions as those during deposition. Annealing is interesting in that it decreases the conductivity of the Cu_{0.66}Cr_{1.33}O₂ layer while it increases the surface roughness and thereby the surface area. The latter increases the sensitivity of the delafossite-type copper oxide layer and thereby of the gas sensor.

The following table 1 shows the changes in conductivity, average roughness, surface coverage and surface area differential, obtained by Atomic Force Microscopy caused by annealing, for a layer with a thickness of 32 nm.

TABLE 1
(layer's thickness = 32 nm)

| annealing t° | as deposited | 900° C. | 1050° C. |
|---|---|---|---|
| conductivity S cm⁻¹ | 12 | 7.3 10⁻⁴ | 4.6 10⁻⁷ |
| average roughness nm | 1.8 | 2.1 | 5.5 |
| surface coverage % | 94 | 87 | 71 |
| AFM surface area differential % | 0.9 | 1.2 | 11 |

The following table 2 shows the changes in conductivity, average roughness, surface coverage and surface area differential caused by annealing, for a layer with a thickness of 140 nm.

TABLE 2
(layer's thickness = 140 nm)

| annealing t° | as deposited | 900° C. | 1050° C. |
|---|---|---|---|
| conductivity S cm⁻¹ | 60 | 2.1 10⁻³ | 4.5 10⁻⁷ |
| average roughness nm | 5.6 | 5.3 | 8.4 |
| surface coverage % | 98 | 88 | 82 |
| AFM surface area differential % | 2.4 | 2.9 | 14.7 |

Annealing is therefore particularly of interest for increasing the sensitivity of the gas sensor. Annealing at the 1050° C. is also substantially more advantageous that respect compared with the anneal at 900° C.

Laser annealing can be used for locally annealing the layer of delafossite-type copper oxide and thereby locally modulate the hole carrier concentration.

FIG. 7 comprises top view of samples two samples considered in the above tables 1 and 2. We can observe, for each thickness of 32 and 140 nm the change in morphology of the surface, where the number of cracks per surface unit increases with annealing and with annealing temperature, resulting in a roughness increase, a surface coverage decrease and a surface area differential increase.

1.-19. (canceled)

20. A gas sensor being an ozone sensor, said sensor comprising:
a substrate;
a layer of delafossite-type copper oxide on the substrate;
a first electrode and a second electrode, both contacting the delafossite-type copper oxide at distant locations so as to permit an electric current through the delafossite-type copper oxide between the locations when applying a voltage at the first and second electrodes;
wherein the delafossite-type copper oxide is Cu_{0.66}Cr_{1.33}O₂.

21. The gas sensor according to claim 20, wherein the delafossite-type copper oxide shows a conductivity that increases when in contact with ozone.

22. The gas sensor according to claim 20, wherein the gas sensor is configured for operating at a constant temperature comprised between 25° and 150° C.

23. The gas sensor according to claim 20, wherein the delafossite-type copper oxide shows an outer surface average roughness of at least 1 nm.

24. The gas sensor according to claim 20, wherein the delafossite-type copper oxide shows an outer surface average roughness of not more than the layer thickness.

25. The gas sensor according to claim 20, wherein the layer of delafossite-type copper oxide shows an average thickness e of at least 20 nm.

26. The gas sensor according to claim 20, wherein the layer of delafossite-type copper oxide shows an average thickness e of not more than 200 nm.

27. The gas sensor according to claim 20, wherein the delafossite-type copper oxide is annealed.

28. The gas sensor according to claim 20, further comprising a heating plate provided on the substrate opposite to the layer of delafossite-type copper oxide.

29. The gas sensor according to claim 20, wherein the substrate is a dielectric layer of a microheater system.

30. The gas sensor according to claim 20, wherein the gas sensor is configured for detecting ozone in a concentration range from 10 to 100 000 ppb.

31. A method of measuring the presence and/or concentration of ozone, said method comprising
using a gas sensor with a substrate, a layer of delafossite-type copper oxide on the substrate, and electrodes on the delafossite-type copper oxide at distant locations,
contacting the delafossite-type copper oxide with the ozone, and
electrically measuring a variation of resistance of the delafossite-type copper oxide;
wherein the electrodes comprise a first electrode and a second electrode, both contacting the delafossite-type copper oxide at distant locations so as to permit an electric current through the delafossite-type copper oxide between the locations when applying a voltage at the first and second electrodes; and
wherein the delafossite-type copper oxide is Cu_{0.66}Cr_{1.33}O₂.

32. The method according to claim 31, wherein during contacting and measuring, the layer of delafossite-type copper oxide is maintained at a temperature comprised between 25° and 150° C.

33. The method according to claim 31, further comprising determining a slope of the variation of resistance of the delafossite-type copper oxide and deducting from the slope the concentration of ozone.

34. The method according to claim 33, wherein the slope of the variation of resistance of the delafossite-type copper oxide is determined over a period of time comprised between 20 and 200 seconds from contacting the delafossite-type copper oxide with the ozone.

35. A method for manufacturing a gas sensor, said method comprising the following steps:
providing a substrate;
depositing a layer of delafossite-type copper oxide on the substrate;
depositing electrodes on the delafossite-type copper oxide;
wherein the electrodes comprise a first electrode and a second electrode, both contacting the delafossite-type copper oxide at distant locations so as to permit an electric current through the delafossite-type copper oxide between the locations when applying a voltage at the first and second electrodes; and
wherein the delafossite-type copper oxide is Cu_{0.66}Cr_{1.33}O₂.

36. The method according to claim 35, comprising the further step of, after depositing the layer of delafossite-type copper oxide on the substrate:
annealing the layer of delafossite-type copper oxide.

37. The method according to claim 36, wherein the annealing step is carried out at a temperature comprised between 800° and 1200° C.

38. The method according to claim 36, wherein the annealing step is carried out by laser scanning.
